(19) United States
(12) Patent Application Publication
(10) Pub. No.: US 2025/0216353 A1
(43) Pub. Date: Jul. 3, 2025

(54) METHOD FOR EVALUATING CONDUCTIVE MATERIAL FOR USE IN RECHARGEABLE BATTERY

(71) Applicants: TOYOTA BATTERY CO., LTD., Kosai-shi (JP); ARTIENCE CO., LTD., Tokyo (JP); TOYOCOLOR CO., LTD., Tokyo (JP)

(72) Inventors: Hidenori YOKOO, Toyohashi-shi (JP); Shotaro DEGUCHI, Toyohashi-shi (JP); Naoki DEGUCHI, Tokyo (JP); Shumpei ONISHI, Tokyo (JP); Risa OKUMURA, Tokyo (JP)

(73) Assignees: TOYOTA BATTERY CO., LTD., Kosai-shi (JP); ARTIENCE CO., LTD., Tokyo (JP); TOYOCOLOR CO., LTD., Tokyo (JP)

(21) Appl. No.: 18/987,565

(22) Filed: Dec. 19, 2024

(30) Foreign Application Priority Data
Dec. 28, 2023 (JP) 2023-222452

(51) Int. Cl. G01N 27/04; G01N 33/00; H01M 4/02; H01M 4/62

(52) U.S. Cl. CPC G01N 27/041; G01N 33/0078; H01M 2004/021; H01M 4/625

(57) ABSTRACT

A method for evaluating a conductive material for use in a rechargeable battery is provided. The rechargeable battery includes an electrode plate in which a mixture layer, containing an active material and the conductive material, is formed on a substrate. The method includes preparing a paste containing simulated primary particles and the conductive material. The simulated primary particles are formed of an insulative material that simulates the active material of the rechargeable battery. The method further includes applying the prepared paste to a simulated substrate that simulates the substrate of the rechargeable battery, drying the applied paste to prepare a test coating containing the simulated primary particles, and measuring a coating resistance R_{S} (Ω·cm) to evaluate the conductive material. The coating resistance corresponds to a surface resistance of the test coating.

### CROSS-REFERENCE TO RELATED APPLICATIONS

This application is based upon and claims the benefit of priority from prior Japanese Patent Application No. 2023-222452, filed on Dec. 28, 2023, the entire contents of which are incorporated herein by reference.

### BACKGROUND

### 1. Field

The following description relates to a method for evaluating a conductive material for use in a rechargeable battery, and more particularly, a method for evaluating a conductive material for use in a rechargeable battery that accurately evaluates conductivity and dispersibility of the conductive material.

### 2. Description of Related Art

Battery electric vehicles, such as hybrid electric vehicles, are powered by rechargeable batteries, for example, lithium-ion rechargeable batteries that are high in voltage, volume energy density (Wh/L), and weight energy density (Wh/kg). In such battery electric vehicles or the like, large currents are discharged during high-load operations and recharged by quick charging or regenerative braking. However, a binder used as a cathode active material has a relatively low conductivity. Such a binder carries a lithium transition metal oxide, for example, lithium nickel cobalt manganese oxide (LiNi_{1/3}Co_{1/3}Mn_{1/3}O₂). In order to improve input/output performance of the lithium-ion rechargeable battery at a high current, a conductive material having a relatively high conductivity may be added to a cathode mixture layer of a cathode plate so as to lower the electric resistance between a large number of particles of the cathode active material and a non-aqueous electrolyte solution or the like. An example of such a conductive material having a relatively high conductivity includes a fibrous carbon material, for example, carbon nanotubes (CNT) or granular acetylene black (AB). In particular, even a relatively small amount of fibrous carbon nanotubes readily forms a conductive network between the cathode active material particles, which are formed of the lithium transition metal oxide dispersed in the cathode mixture layer. This lowers the electric resistance between the non-aqueous electrolyte solution and the cathode active material.

Japanese Laid-Open Patent Publication No. 2015-150515 describes an example of carbon nanotubes that are readily dispersed in resin and demonstrate a relatively high conductivity in the resin. Such carbon nanotubes are added to the cathode mixture layer so as to lower the electric resistance (coating resistance) in the cathode mixture layer.

### SUMMARY

The lithium transition metal oxide used as the cathode active material has primary particles and secondary particles. Specifically, crystalline primary particles are first prepared, and then aggregated to form spherical secondary particles that each have a cavity. This allows for efficient reactions on the surface of the cathode active material particles. The secondary particle of the cathode active material includes gaps formed between the primary particles. The gaps extend through the surface of the secondary particle and are continuous with the internal cavity. The dimensions of the gaps vary greatly depending on firing conditions or the like.

Typically, a coating resistance of a cathode mixture layer of a cathode plate is measured by actually forming a cathode mixture layer on a substrate, which is formed by a polyethylene terephthalate (PET) film or the like, and then performing measuring on the surface. In this manner, the conductivity of the cathode mixture layer is evaluated as the coating resistance. However, the coating resistance changes when the conductive material included in the cathode mixture layer enters into the cathode active material particles through the gaps, unlike the case in which a conductive material is dispersed in a homogeneous resin as described in Patent Literature 1. For example, when the gaps in the cathode active material particles are relatively large, a relatively large amount of conductive material may enter into the particles, which in turn, decreases the amount of conductive material present between the cathode active material particles. This may hinder formation of a conductive network by the conductive material and increase the resistance. For this reason, even when the coating resistance of the cathode mixture layer is measured accurately, the conductive material may be evaluated incorrectly.

Such a problem of incorrect evaluation of the conductive material is not limited to the hollow particles of the cathode active material in the cathode plate, and may also occur in an anode plate. Furthermore, this problem is not limited to lithium-ion rechargeable batteries and may also occur in other types of non-aqueous electrolyte solution rechargeable batteries, as well as in other types of rechargeable batteries, such as alkaline rechargeable batteries.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method for evaluating a conductive material for use in a rechargeable battery is provided. The rechargeable battery includes an electrode plate in which a mixture layer, containing an active material and the conductive material, is formed on a substrate. The method includes preparing a paste containing simulated primary particles and the conductive material. The simulated primary particles are formed of an insulative material that simulates the active material of the rechargeable battery. The method further includes applying the prepared paste to a simulated substrate that simulates the substrate of the rechargeable battery, drying the applied paste to prepare a test coating containing the simulated primary particles, and measuring a coating resistance R_{S} (Ω·cm) to evaluate the conductive material. The coating resistance corresponds to a surface resistance of the test coating.

In the above evaluation method, a mixture volume ratio Rᵥ (vol %) of the conductive material to the simulated primary particles in the paste may be set in accordance with a mixture volume ratio Rᵥ (vol %) of the conductive material to the active material.

In the above evaluation method, a mixture mass ratio R_{W} (wt %) of the conductive material to the simulated primary particles may be set in accordance with a range of a graph, the graph showing the coating resistance R_{S} (Ω·cm) of the test coating and changes in the mixture mass ratio R_{W} (wt %), the range including a part at which a curvature of the graph is maximum.

In the above evaluation method, when a mixture mass ratio of the conductive material to the simulated primary particles is represented by R_{W} (wt %), the mixture mass ratio R_{W} (wt %) may be between 1 wt % and 3 wt %, inclusive.

In the above evaluation method, an average particle diameter D_{S} (d50) (μm) of the simulated primary particles may be substantially the same as that of particles of the active material of the rechargeable battery. In this case, an average particle diameter D_{S} (d50) (μm) of the simulated primary particles may be between 0.1 μm and 50 μm, inclusive.

In the above evaluation method, the test coating may have a thickness (μm) that is the same as a thickness (μm) of the mixture layer of the rechargeable battery.

In the above evaluation method, the active material may include secondary particles formed by aggregated primary particles. In the above evaluation method, the conductive material may be formed of a fibrous carbon. Further, in the above evaluation method, the simulated primary particles may be formed of alumina.

In the above evaluation method, the simulated substrate may be formed of an insulative material.

In the above evaluation method, the simulated substrate may be formed by a PET film.

In the above evaluation method, an average diameter D_{C} (d50) (nm) of the conductive material may be between 1 nm and 100 nm, inclusive. In the above evaluation method, an average length L_{C} (d50) (nm) of the conductive material may be between 100 nm and 10000 nm, inclusive.

In the above evaluation method, the rechargeable battery may be a lithium-ion rechargeable battery. Further, in the above evaluation method, the electrode plate may be a cathode plate.

Other features and aspects will be apparent from the following detailed description, the drawings, and the claims.

### BRIEF DESCRIPTION OF THE DRAWINGS

FIG. 1 is a schematic diagram illustrating a perspective view of an enlarged cathode active material.

FIG. 2 is a schematic diagram illustrating a cross section of the enlarged cathode active material.

FIG. 3A is a schematic diagram illustrating a relationship of the cathode active material having almost no gaps and a conductive material. FIG. 3B is a schematic diagram illustrating a relationship of the cathode active material having a moderate number of gaps and the conductive material. FIG. 3C is a schematic diagram illustrating a relationship of the cathode active material having a relatively large number of gaps and the conductive material.

FIG. 4 is a schematic cross-sectional diagram illustrating variations of the cathode active material in a cathode mixture layer.

FIG. 5A is a schematic diagram illustrating a simulated cathode plate in accordance with an embodiment. FIG. 5B is a schematic diagram illustrating a cathode plate of a lithium-ion rechargeable battery in accordance with the embodiment.

FIG. 6 is a schematic diagram illustrating a relationship of simulated primary particles and the conductive material.

FIG. 7 is a schematic diagram illustrating a perspective view of the lithium-ion rechargeable battery in accordance with the present embodiment.

FIG. 8 is a schematic diagram illustrating an electrode body in an unrolled state.

FIG. 9 is a table showing conditions of experimental examples.

FIG. 10 is a diagram illustrating coefficients of variation CV indicating variability of the cathode active material and the simulated primary particles.

FIG. 11 is a graph showing a relationship between a coating resistance R_{S} (Ω·cm) of a test coating and changes in a mixture mass ratio R_{W} (wt %) of the conductive material to the simulated primary particles.

FIG. 12 is a graph enlarging part of the graph shown in FIG. 11.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

This description provides a comprehensive understanding of the methods, apparatuses, and/or systems described. Modifications and equivalents of the methods, apparatuses, and/or systems described are apparent to one of ordinary skill in the art. Sequences of operations are exemplary, and may be changed as apparent to one of ordinary skill in the art, with the exception of operations necessarily occurring in a certain order. Descriptions of functions and constructions that are well known to one of ordinary skill in the art may be omitted.

Exemplary embodiments may have different forms, and are not limited to the examples described. However, the examples described are thorough and complete, and convey the full scope of the disclosure to one of ordinary skill in the art.

In this specification, “at least one of A and B” should be understood to mean “only A, only B, or both A and B.”

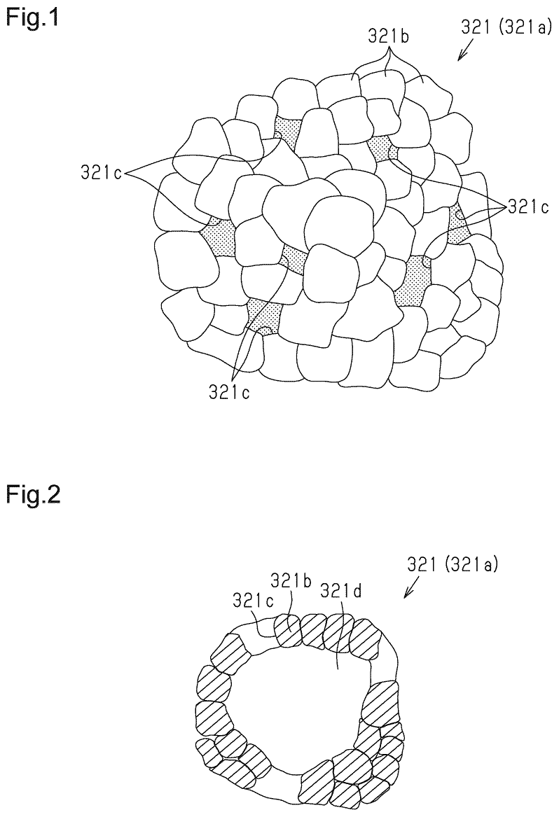

A method for evaluating a conductive material for use in a rechargeable battery according to the present disclosure will now be described with reference to FIGS. 1 to 12, using an example embodiment of a method for evaluating carbon nanotubes (CNT). The CNT serves as a conductive material 322 of a cathode plate 3 for use in a lithium-ion rechargeable battery 1.

The present embodiment is merely an example, and an electrode plate is not limited to a cathode plate. Furthermore, a rechargeable battery is not limited to a lithium-ion rechargeable battery.

### Overview of the Present Embodiment

### Background Art of the Present Embodiment

FIG. 1 is a schematic diagram illustrating a perspective view of an enlarged cathode active material 321. FIG. 2 is a schematic diagram illustrating a cross section of the enlarged cathode active material 321. As shown in FIGS. 1 and 2, a lithium transition metal oxide serving as the cathode active material 321 is formed by first preparing crystalline primary particles 321b. The lithium transition metal oxide in such a state is prone to aggregation. In order to improve dispersibility, the primary particles 321b are aggregated to form spherical secondary particles 321a that each have a cavity 321d. This allows for efficient reaction on the surface of the cathode active material 321. Such a cathode active material 321 having the cavity 321d inside the secondary particle 321a includes gaps 321c. The gaps 321c extend through the surface of the secondary particle 321a and are continuous with the cavity 321d. The number and area of the gaps 321c vary greatly depending on firing conditions or the like.

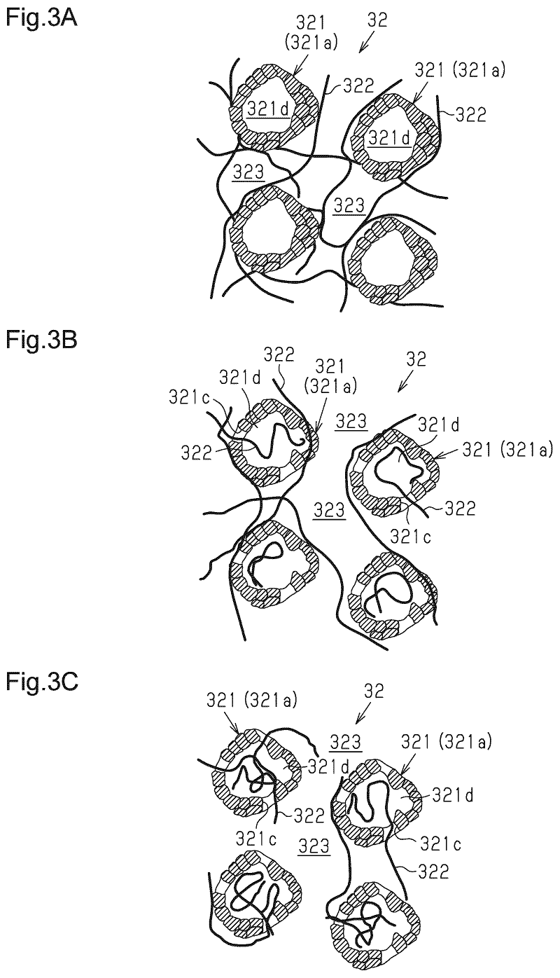

FIG. 3A is a schematic diagram illustrating a relationship of the cathode active material 321 having almost no gaps and the conductive material 322. FIG. 3B is a schematic diagram illustrating a relationship of the cathode active material 321 having a moderate number of gaps and the conductive material 322. FIG. 3C is a schematic diagram illustrating a relationship of the cathode active material 321 having a relatively large number of gaps and the conductive material 322. The relationship of the cathode active material 321 and the conductive material 322 varies greatly depending on the gaps 321c. For example, as shown in FIG. 3A, when there are almost no gaps 321c, the conductive material 322 does not enter the cavity 321d of the cathode active material 321. In this case, the conductive material 322 is present in a binder 323 between adjacent secondary particles 321a of the cathode active material 321. This increases the density of the conductive material 322 per unit volume of a cathode mixture layer 32 when the amount (vol %) of the conductive material 322 contained in the cathode mixture layer 32 is the same. As a result, the fibers of the conductive material 322 readily contact one another and form a conductive network. This decreases a coating resistance R_{S} (Ω·cm) of the cathode mixture layer 32.

On the other hand, as shown in FIG. 3C, when there are a relatively large number of gaps 321c, the conductive material 322 easily enters the cavity 321d of the cathode active material 321 through the gaps 321c. In this case, the conductive material 322 is present in the binder 323 between adjacent secondary particles 321a of the cathode active material 321 and also inside the cavity 321d. This decreases the amount of the conductive material 322 present per unit volume of the cathode mixture layer 32 when the amount (vol %) of the conductive material 322 contained in the cathode mixture layer 32 is the same. As a result, the fibers of the conductive material 322 are less likely to contact one another to form a conductive network. This increases the coating resistance R_{S} (Ω·cm) of the cathode mixture layer 32.

As shown in FIG. 3B, when the gaps 321c are smaller in number and area than those in FIG. 3C, the properties of such gaps 321c will be between those shown in FIGS. 3A and 3C.

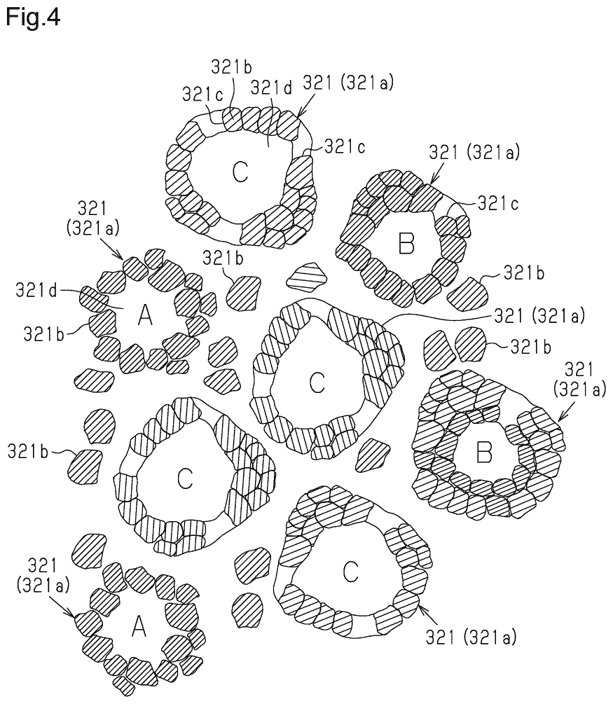

FIG. 4 is a schematic cross-sectional diagram illustrating variations of the cathode active material 321 in the cathode mixture layer 32. As shown in FIG. 4, the cathode active material 321 serving as an actual raw material is not homogeneous and includes gaps 321c, such as those shown in FIGS. 3A to 3C. The example of FIG. 4 includes the cathode active materials 321 shown in FIGS. 3A to 3C; specifically, two particles of the cathode active material 321 shown in FIG. 3A, two particles of the cathode active material 321 shown in FIG. 3B, and four particles of the cathode active material 321 shown in FIG. 3C. In FIG. 4, “A” corresponds to the cathode active material 321 shown in FIG. 3A, “B” corresponds to the cathode active material 321 shown in FIG. 3B, and “C” corresponds to the cathode active material 321 shown in FIG. 3C.

Further, in the actual cathode mixture layer 32, a cathode mixture paste of the cathode mixture layer 32 is applied to a cathode substrate 31 and then pressed in a pressing step. This may collapse some secondary particles 321a of the cathode active material 321 or deform some secondary particles 321a into separate primary particles 321b. Therefore, the cathode active material 321 in a completed lithium-ion rechargeable battery 1 will not be homogeneous. Accordingly, no matter how accurately the coating resistance R_{S} (Ω·cm) of the cathode mixture layer 32 is measured, the conductivity of the conductive material 322 or the overall quality of the cathode mixture layer 32 may not be evaluated appropriately.

### Cathode Plate 3 and Simulated Cathode Plate 103 of Lithium-Ion Rechargeable Battery 1

FIG. 5A is a schematic diagram illustrating a simulated cathode plate 103 in accordance with the present embodiment. FIG. 5B is a schematic diagram illustrating the cathode plate 3 of the lithium-ion rechargeable battery 1 in accordance with the present embodiment. The simulated cathode plate 103 is used in tests to accurately measure the conductivity and dispersibility of the conductive material 322, which cannot be accurately obtained using the cathode plate 3 of the lithium-ion rechargeable battery 1.

### Structure of Cathode Plate 3 of Lithium-Ion Rechargeable Battery 1

First, the cathode plate 3 of the lithium-ion rechargeable battery 1 in accordance with the present embodiment will be described with reference to FIG. 5B. In the lithium-ion rechargeable battery 1 of the present embodiment, the cathode plate 3 includes the cathode substrate 31 formed by an Al foil, and the cathode mixture layer 32 formed on the cathode substrate 31. The cathode mixture layer 32 is a layer formed by applying the cathode mixture paste to the cathode substrate 31 and drying and pressing the paste. The cathode mixture paste is prepared by kneading the cathode active material 321, the conductive material 322, and the binder 323 with a solvent.

In such a cathode plate 3, the conductive network formed by the conductive material 322 varies in accordance with the shapes of the secondary particles 321a of the cathode active material 321, and particularly, in accordance with the number of gaps 321c. A method for evaluating the conductive material 322 will now be described, together with a description of the term “coating resistance R_{S} (Ω·cm)” as used in the present embodiment. When measuring a coating resistance R_{S} (Ω·cm) in the present embodiment, first, the cathode mixture layer 32 is applied to the simulated substrate 131 formed by a polyethylene terephthalate (PET) film, instead of the cathode substrate 31 formed by an Al foil. Then, two measurement points MP1 and MP2 are set on the surface of the cathode mixture layer 32 at positions separated from each other by 1 cm. Probes of a resistance meter OM are brought into contact with the measurement points MP1 and MP2 to measure the coating resistance R_{S} (Ω·cm). The coating resistance corresponds to the surface resistance of the conductive material 322 between the measurement points MP1 and MP2. For example, the measurement is performed by a four-terminal method using a four-probe resistance meter manufactured by HIOKI E.E. CORPORATION. Such a coating resistance R_{S} (Ω·cm) allows for accurate evaluation of the conductivity and dispersibility of the conductive material.

Conventionally, the cathode substrate 31 formed by a conductive Al foil is disposed on a surface of the cathode mixture layer 32 facing the surface on which the measurement points MP1 and MP2 are arranged. Therefore, in actuality, only the resistance of the cathode mixture layer 32 in a thickness direction is obtained. Accordingly, it is difficult to accurately evaluate the conductive material 322 used. In this respect, the cathode substrate 31 formed by an Al foil is replaced with the simulated substrate 131 formed by a polyethylene terephthalate (PET) film so as to measure the coating resistance R_{S} (Ω·cm) without being affected by the cathode substrate 31 formed of an Al foil.

However, as described above, the secondary particles 321a of the cathode active material 321 are also conductive. Also, the shapes of the secondary particles 321a of the cathode active material 321, and particularly, the number of gaps 321c affect the conductive network formed by the conductive material 322. This hinders appropriate observations of the conductivity of the conductive material 322 or the conductive network formed through dispersion of the conductive material 322.

### Structure of Simulated Cathode Plate 103 of the Present Embodiment

The simulated cathode plate 103 of the present embodiment will now be described with reference to FIG. 5A. As shown in FIG. 5A, in the simulated cathode plate 103 of the present embodiment, the cathode active material 321 is replaced with simulated primary particles 132a.

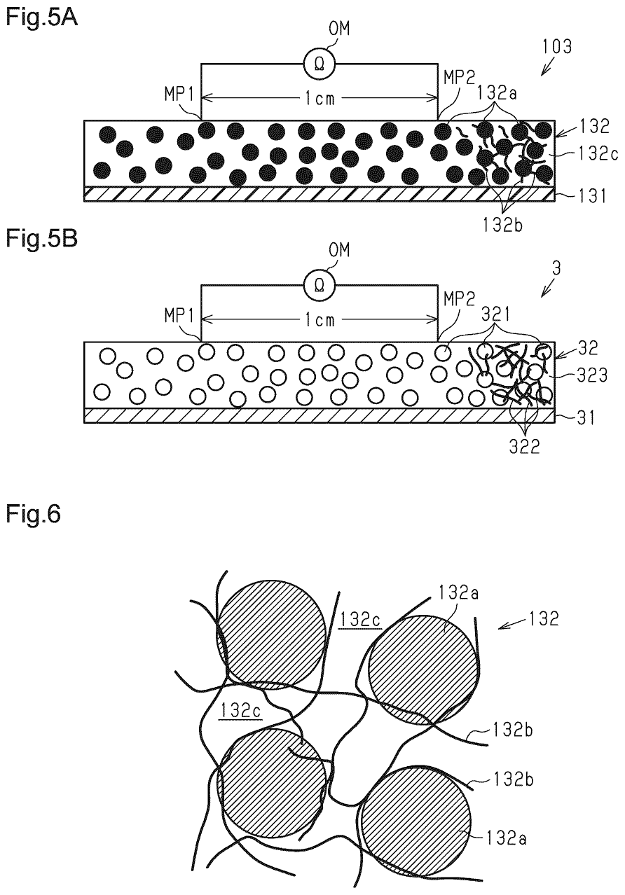

FIG. 6 is a schematic diagram illustrating a relationship of the simulated primary particles 132a and a conductive material 132b. As shown in FIG. 6, the simulated primary particles 132a are formed of an insulative material that simulates the cathode active material 321 of the lithium-ion rechargeable battery 1, such as that shown in FIG. 3A. The term “simulated” refers to a structure having an external form similar to that of the secondary particle 321a of the cathode active material 321 to imitate the cathode active material 321. For example, an average particle diameter (d50) (μm) is substantially the same. Thus, the average particle diameter D_{S} (μm) of the simulated primary particles 132a is substantially the same as that of the secondary particle of the active material of the rechargeable battery (in the present embodiment, the lithium-ion rechargeable battery). In this specification, unless otherwise specified, “average particle diameter D_{S} (μm) of simulated primary particles 132a” refers to the median diameter (d50) in a frequency distribution obtained by laser diffraction. An average diameter D_{C} (nm) and an average length L_{C} (nm) of the conductive material 132b are values obtained by image analysis of electron micrographs.

The term “substantially” means that even when there are some differences or is unevenness in shape, the mechanical function of the simulated primary particles 132a in a test coating 132 is equivalent to that of the cathode active material 321 in the cathode mixture layer 32. The simulated primary particles 132a of the present example are formed of an insulative material, specifically, particles of alumina. Therefore, the electrochemical function of the simulated primary particles 132a differs from that of the cathode active material 321.

Further, the term “primary particles” refers to particles that are in an initial state when manufacturing certain particles. Each of the simulated primary particles 132a is a solid crystal. In contrast, in the cathode active material 321, a large number of the primary particles are aggregated to form a single secondary particle 321a. As a result, the secondary particles 321a of the cathode active material 321, such as those shown in FIGS. 3B and 3C, each have the cavity 321d and the gaps 321c. On the other hand, the simulated primary particles 132a do not include such internal cavities or gaps.

### Conductive Material 132b

Although the conductive material 132b and the conductive material 322 of the cathode mixture layer have different reference numerals, these two are the same materials. Specifically, the carbon nanotubes, described as an example of the conductive materials in the present embodiment, are identical in type, length, diameter, mass, added amount, and the like. A mixture volume ratio R_{V} (vol %) of the conductive material 132b to the simulated primary particles 132a in a test coating paste is set in accordance with a mixture volume ratio R_{V} (vol %) of the conductive material 322 to the cathode active material 321. Thus, the amount of the conductive material 132b will be substantially the same as the amount of the conductive material 322. The mixture volume ratio R_{V} (vol %) may be replaced in advance by a mixture mass ratio R_{W} (wt %) of the conductive material 132b to the simulated primary particles 132a. This is because an accurate volume (mm³) is difficult to obtain due to the effects of bulk density and porosity, and a mass (g) may be measured relatively easily.

The method for evaluating the conductive material 322 for use in the lithium-ion rechargeable battery 1 of the present embodiment is designed to accurately evaluate the conductivity and dispersibility of the conductive material 322 contained in the cathode mixture layer 32 of the lithium-ion rechargeable battery 1. Therefore, the actual state of the conductive material 322 is precisely reproduced as described above. In the field of engineering, a conductivity is typically expressed in unit of (S/m). However, in the present embodiment, the measurement described above is performed to analyze the dispersion state or the like of the actual conductive material 322 in the lithium-ion rechargeable battery 1.

In the present embodiment, an example of the conductive material 322, 132b includes a fibrous carbon, specifically, carbon nanotubes (CNT). Instead, the conductive material 322, 132b may be other types of conductive material, for example, carbon microfibers, granular acetylene black (AB), or the like. In the same manner as a fibrous conductive material, a granular conductive material may also enter the cavity 321d through the gaps 321c of the cathode active material 321 or enter spaces between the primary particles of the cathode active material 321. Accordingly, in the same manner as a fibrous conductive material, a granular conductive material also affects formation of the conductive network by the conductive material in the binder 323.

In the present embodiment, specifically, the average diameter D_{C} (d50) (nm) of the conductive material 132b is between 1 nm and 100 nm, inclusive. The average length L_{C} (d50) (nm) of the conductive material is between 100 nm and 10000 nm, inclusive.

### Simulated Substrate 131

The simulated cathode plate 103 of the present embodiment has the same appearance as the cathode substrate 31. However, the simulated cathode plate 103 includes the simulated substrate 131 formed of an insulative material, instead of an Al foil. Thus, the simulated electrode plate (in the present embodiment, the simulated cathode electrode plate 103) including the simulated substrate 131 and a test coating 132 has the same appearance as the electrode plate (in the present embodiment, the cathode substrate 31) of the rechargeable battery (in the present embodiment, the lithium-ion rechargeable battery). Any material may be used for the simulated substrate 131 as long as the material is insulative and allows for application of a test coating paste. The present example uses a polyethylene terephthalate (PET) film that has a relatively high insulating property and facilitates application of a test coating paste. The test coating 132 is formed on the simulated substrate 131. The test coating 132 is a layer formed by applying a test coating paste to the simulated substrate 131, and drying and pressing the applied paste. The test coating paste is prepared by kneading the simulated primary particles 132a, the conductive material 132b, and the binder 132c with a solvent. The solvent for the conductive material 132b and the binder 132c is the same as the solvent for the conductive material 322 and the binder 323 of the cathode mixture paste. In other words, the test coating paste only differs from the cathode mixture paste in which the cathode active material 321 is replaced by the simulated primary particles 132a.

### Measurement of Coating Resistance R_{S} (Ω·cm)

In the same manner as the typical measurement method, the method for evaluating the conductive material for use in the lithium-ion rechargeable battery 1 of the present embodiment is performed on a surface of the test coating 132 in a completed simulated cathode plate 103. Measurement points MP1 and MP2 are set at positions separated from each other by 1 cm, and then probes of a resistance meter OM are brought into contact with the measurement points MP1 and MP2 to measure the coating resistance R_{S} (Ω·cm) of the conductive material therebetween.

The cathode active material 321 has already been replaced with the insulative simulated primary particles 132a. Also, the cathode substrate 31 has already been replaced with the insulative simulated substrate 131. The simulated primary particles 132a are insulative and include practically no gaps 321c. Thus, the density of the conductive material 322 remains constant. Accordingly, the state of the conductive network formed by the conductive material 322 will always be the same. This allows for accurate observations of the conductivity of the conductive material 322, the state of the conductive network formed by the dispersed conductive material 322, and the like. Further, the simulated substrate 131, which is formed of an insulative resin, is disposed on a surface of the cathode mixture layer 32 facing the surface on which the measurement points MP1 and MP2 are arranged. Therefore, only the resistance of the conductive network formed by the conductive material 132b in the test coating 132 may be measured without being affected by the conductive cathode substrate 31. This allows for accurate evaluation of the conductive material 132b used as a raw material.

### Structure of the Present Embodiment

An example of the lithium-ion rechargeable battery 1 of the present embodiment will now be described. This lithium-ion rechargeable battery 1 serves as the basis for the evaluation method of CNT, which is the conductive material 322 for use in the lithium-ion rechargeable battery 1. This description is not intended to limit the battery type.

### Structure of Lithium-Ion Rechargeable Battery 1

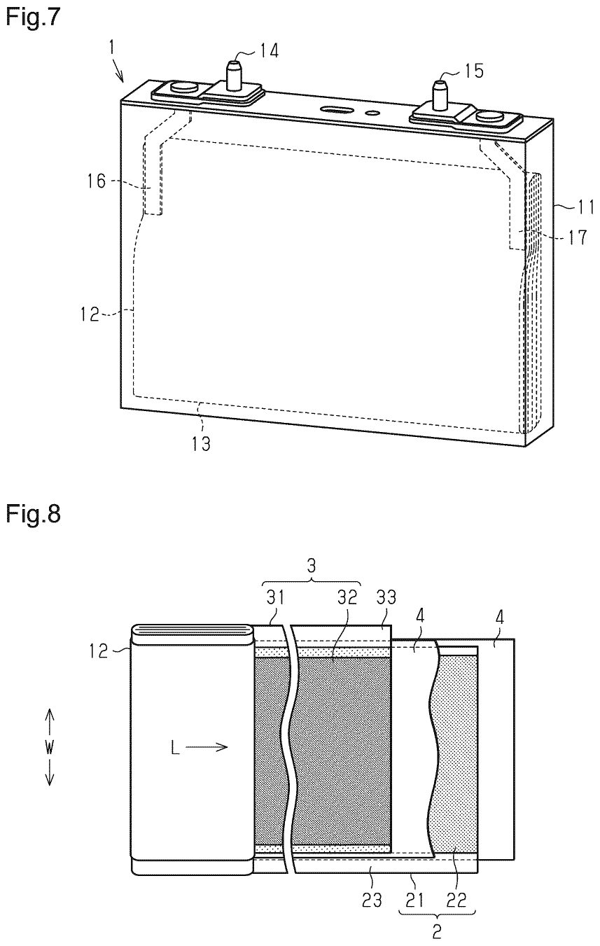

FIG. 7 is a schematic diagram illustrating a perspective view of the lithium-ion rechargeable battery 1 in accordance with the present embodiment.

As shown in FIG. 7, the lithium-ion rechargeable battery 1 is a battery cell that forms a battery module of a battery pack mounted on a vehicle. The lithium-ion rechargeable battery 1 includes a box-shaped battery case 11 having an open upper end. The battery case 11 accommodates an electrode body 12. The battery case 11 is filled with a non-aqueous electrolyte solution 13 injected through a liquid injection hole. The battery case 11 is formed from a metal such as an aluminum alloy, and forms a sealed battery container by attaching a lid. Further, the lithium-ion rechargeable battery 1 includes a cathode external terminal 14 and an anode external terminal 15 used for charging and discharging the lithium-ion rechargeable battery 1. The cathode external terminal 14 is electrically connected through the lid to a cathode current collector terminal 16 inside the battery case 11. The anode external terminal 15 is electrically connected through the lid to an anode current collector terminal 17 inside the battery case 11. The cathode current collector terminal 16 is electrically connected to a cathode current collector 33 (refer to FIG. 8) of the electrode body 12. The anode current collector terminal 17 is electrically connected to an anode current collector 23 (refer to FIG. 8) of the electrode body 12.

### Electrode Body 12

FIG. 8 is a schematic diagram illustrating the electrode body 12 in an unrolled state. In the electrode body 12, an anode plate 2 and the cathode plate 3 are stacked with a separator 4 arranged in between. The stack of the anode plate 2, the cathode plate 3, and the separator 4 is rolled and flattened. The anode plate 2 includes an anode mixture layer 22 formed on an anode substrate 21. The anode substrate 21 is formed by a copper foil, which serves as a base material. The anode current collector 23 is arranged at one end of the electrode body 12 in a widthwise direction W (rolling axial direction) that is orthogonal to a direction in which the anode substrate 21 is rolled (rolling direction L). The anode current collector 23 corresponds to where the anode mixture layer 22 is not formed such that the anode substrate 21 is exposed.

The cathode plate 3 includes the cathode mixture layer 32 formed on the cathode substrate 31. The cathode substrate 31 is formed by an aluminum foil, which serves as a base material. As shown in FIG. 8, the cathode current collector 33 is arranged at the other end of the electrode body 12 (opposite to anode current collector 23) in the widthwise direction W (rolling axial direction) that is orthogonal to the direction in which the cathode substrate 31 is rolled (rolling direction L). The cathode current collector 33 corresponds to where the cathode mixture layer 32 is not formed such that the metal of the cathode substrate 31 is exposed.

### Stack Structure of Electrode Body 12

As shown in FIG. 8, the basic structure of the electrode body 12 of the lithium-ion rechargeable battery 1 includes the anode plate 2, the cathode plate 3, and the separators 4.

The anode plate 2 includes the anode mixture layer 22 disposed on two opposite surfaces of the anode substrate 21, which serves as an anode base member. One end of the anode substrate 21 corresponds to the anode current collector 23 where the metal is exposed.

The cathode plate 3 includes the cathode mixture layers 32 disposed on two opposite surfaces of the cathode substrate 31, which serves as a cathode base member. One end of the cathode substrate 31 located at the side opposite to the anode current collector 23 corresponds to the cathode current collector 33 where the metal is exposed.

The stack of the anode plate 2 and the cathode plate 3 is formed with the separator 4 disposed in between. The stack is rolled about the rolling axis in a longitudinal direction as shown in FIG. 8, and then flattened to form the roll-type electrode body 12 such as that shown in FIG. 7.

### Non-Aqueous Electrolyte Solution 13

The non-aqueous electrolyte solution 13 of the lithium-ion rechargeable battery 1 of the present embodiment shown in FIG. 7 is a composition in which a lithium salt is dissolved in an organic solvent. The lithium salt may be LiClO₄, LiPF₆, LiAsF₆, LiBF₄, LiSO₃CF₃ or the like. Examples of the organic solvent include a cyclic carbonate, such as ethylene carbonate, propylene carbonate, butylene carbonate, or trifluoropropylene carbonate; a chain carbonate, such as diethyl carbonate, dimethyl carbonate, ethyl methyl carbonate, or dipropyl carbonate; an ether compound, such as tetrahydrofuran, 2-methyltetrahydrofuran, or dimethoxyethane; a sulfur compound, such as ethyl methyl sulfone or butane sultone; and a phosphorus compound, such as triethyl phosphate or trioctyl phosphate. One of these compounds or a mixture of more than one of these compounds may be used as the non-aqueous electrolyte solution 13. The composition of the non-aqueous electrolyte solution 13 is not limited to those described above.

In the present embodiment, ethylene carbonate (EC) is used as the organic solvent.

In the present embodiment, for example, lithium bis(oxalate)borate (LiBOB, LiB(C₂O₄)₂) or the like may be added as a film forming agent.

### Component of Electrode Body 12

The components of the electrode body 12, namely, the anode plate 2, the cathode plate 3, and the separator 4, will now be described.

### Anode Plate 2

The anode plate 2 includes the anode mixture layers 22 formed on the two opposite surfaces of the anode substrate 21, which serves as the anode base member as shown in FIG. 8. In a battery element manufacturing step, an anode mixture paste is applied to the anode substrate 21 to form the anode mixture layer 22. Then, a drying step, a pressing step, and a cutting step are performed to complete the anode plate 2.

In the present embodiment, the anode substrate 21 is formed by a Cu foil. The anode substrate 21 serves as a base for the anode mixture layer 22 and has a functionality of a current collecting member that collects electricity from the anode mixture layer 22. One end of the anode substrate 21 is the anode current collector 23 where the anode mixture layer 22 is not formed such that the metal surface is exposed. This electrically connects anode active material particles to the anode external terminal 15 via the anode substrate 21, the anode current collector 23, and the anode current collector terminal 17.

The anode mixture layer 22 includes an anode active material as a primary material, and a binder, an additive, and the like as secondary materials. The primary material and the secondary materials are mixed with an organic solvent or the like, and kneaded to prepare the anode mixture paste. Such an anode mixture paste is applied to the anode substrate 21. The applied anode mixture paste is dried and pressed to complete the anode plate 2.

In the present embodiment, the anode active material is a powder of graphite particles GP formed from graphite having a layered structure. The anode active material is capable of storing and releasing lithium ions Lit.

### Cathode Plate 3

As shown in FIGS. 5B and 8, the cathode plate 3 includes the cathode substrate 31, serving as the cathode base member, and the cathode mixture layer 32 formed on the cathode substrate 31. In a battery element manufacturing step, the cathode mixture paste is applied to the cathode substrate 31 to form the cathode mixture layer 32. Then, a drying step, a pressing step, and a cutting step are performed to complete the cathode plate 3.

The cathode plate 3 includes the cathode mixture layers 32 formed on the two opposite surfaces of the cathode substrate 31. In the present embodiment, the cathode substrate 31 is formed by an Al foil. The cathode substrate 31 serves as a base for the cathode mixture layer 32 and has a functionality of a current collecting member that collects electricity from the cathode mixture layer 32.

An Al foil is described as an example of the cathode base member that forms the cathode substrate 31. The cathode base member is formed from, for example, a conductive material including a metal having satisfactory electric conduction. The material having satisfactory electric conduction may be, for example, an Al foil or a material including an Al alloy. The configuration of the cathode substrate 31 is not limited to that described above.

The cathode mixture layer 32 is formed by applying the cathode mixture paste to the cathode substrate 31 and drying the paste. The cathode mixture layer 32 includes the secondary particles 321a of the cathode active material 321, the conductive material 322, the binder 323, and an additive of a dispersant or the like.

The cathode active material 321 contains a lithium transition metal oxide having a layered crystal structure. The lithium transition metal oxide includes one or more predetermined transition metal elements in addition to Li. Preferably, the transition metal element included in the lithium transition metal oxide is at least one of Ni, Co, and Mn. The cathode active material 321 of the present embodiment is an example of a ternary type referred to as NCM, which includes all of Ni, Co, and Mn.

The cathode active material 321 of the present embodiment is not limited to a lithium transition metal oxide that includes all of Ni, Co, and Mn. Alternatively, the lithium transition metal oxide may have a composition containing other types of element such as Al.

### Separator 4

The separator 4 is a non-woven fabric of polypropylene, which is a porous resin, or the like that has a superior insulation property and holds the non-aqueous electrolyte solution 13 between the anode plate 2 and the cathode plate 3. Further, the separator 4 may be any one of or a combination of a porous polymer film (e.g., porous polyethylene film, porous polyolefin film, porous polyvinyl chloride film, or the like) and a lithium-ion-conductive or ion-conductive polymer electrolyte film.

### Method for Evaluating Conductive Material for Use in Lithium-Ion Rechargeable Battery 1

Experimental examples for the evaluation method of the conductive material for use in the lithium-ion rechargeable battery 1 of the present embodiment will now be described.

### Simulated Primary Particle 132A

The average particle diameter D (d50) (μm) of the simulated primary particles 132a was substantially the same as that of the particles of the cathode active material 321 in the lithium-ion rechargeable battery 1. Specifically, in the present embodiment, the average particle diameter D (d50) (μm) of the simulated primary particles 132a was between 0.1 μm and 50 μm, inclusive.

### Measurement Condition of Alumina Coating Resistance

A composition of 30 g of the test coating paste included 7.41 g of alumina, 3.96 g of a 3% CNT solution, and 18.63 g of N-methyl-2-pyrrolidone (NMP). The CNT was 0.1188 g, which corresponded to 1.6 wt % of alumina.

The paste was prepared as follows. Specifically, the paste was kneaded as described below. The above materials were agitated in a mixing and defoaming machine at 2000 rpm for thirty seconds, and then the content was stirred with a spatula to visually check for clusters. Found clusters were mashed with the spatula. Then, the content was again agitated in the mixing and defoaming machine at 2000 rpm for five minutes to prepare a slurry for coating. An example of the mixing and defoaming machine includes the non-vacuum THINKY MIXER ARE-312 manufactured by THINKY CORPORATION.

The test coating was prepared as follows. The test coating 132 had a thickness (μm) that is the same as a thickness (μm) of the cathode mixture layer 32 of the lithium-ion rechargeable battery 1. The test coating 132 was prepared by performing an applying step and a drying step as described below. First, 4 to 5 ml of the test coating paste was placed on a PET film, which serves as the simulated substrate 131. Then, the paste was spread using a bar coater and a 300 μm applicator. After the applying step, the paste was heated and dried in a dryer with hot air at 120° C. for fifteen minutes to form the test coating 132. The thickness of the test coating 132 was measured with a coating thickness gauge (digital micrometer). The coating resistance R_{S} (Ω·cm), which corresponds to the surface resistance of the test coating 132, was measured by four-probe sensing.

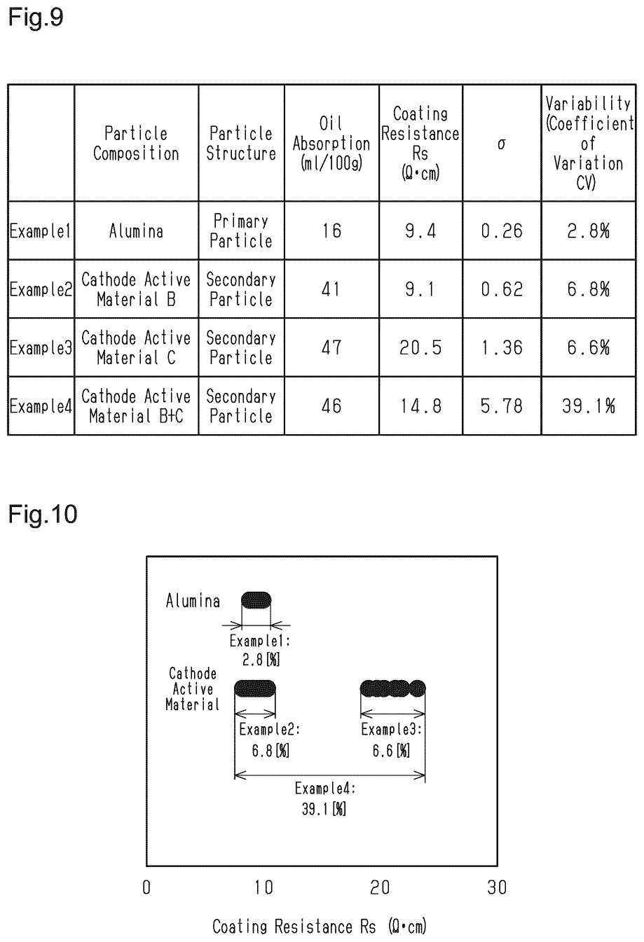

FIG. 9 is a table showing conditions of Experimental Examples 1 to 4. The conditions include whether the particle composition was the simulated primary particles 132a or the cathode active material 321, and whether the particle structure was the primary particles or the secondary particles.

Also, an oil absorption (ml/100 g) was measured. The oil absorption (ml/100 g) indicates an amount of linseed oil absorbed. The linseed oil having a relatively high permeability was used so that the linseed oil permeates the cavity 321d through the gaps 321c. A greater amount of oil absorption (ml/100 g) indicates that the conductive material 322 formed by CNT was more likely to enter the cavity 321d through the gaps 321c.

The conductivity was evaluated as follows. The coating resistance R_{S} (Ω·cm) of the test coating 132 was measured by the method shown in FIG. 5A. Further, the coating resistance R_{S} (Ω·cm) of the cathode mixture layer 32 was measured by the method shown in FIG. 5B. In order to harmonize the measurement conditions, the cathode substrate 31 shown in FIG. 5B was replaced with the simulated substrate 131 shown in FIG. 5A, which is formed from PET. In the table, such measurements are both shown under “Coating Resistance R_{S} (Ω·cm)”.

The symbol “σ” represents a standard deviation, specifically, a standard deviation of the coating resistance R_{S} (Ω·cm).

The “Coefficient of Variation CV” indicating “Variability” was obtained using the following equation: “coefficient of variation CV=standard deviation o/average value”. The coefficient of variation CV is a non-dimensional value used when evaluating variability in multiple data sets expressed in different units or when evaluating a relationship of a data set with respect to an average value. In the present embodiment, the coefficient of variation CV is expressed in percentage (%).

### Examples 1 to 4

Example 1: The simulated primary particles 132a were used, the particle composition was alumina, and the particle structure was solid primary particles. Accordingly, the oil absorption (ml/100 g) was relatively small at 16 ml/100 g. This means that the simulated primary particles 132a of Example 1 had an extremely small amount of cavities 321d and gaps 321c, such as those of the cathode active material 321.

In Example 1, the average value of the coating resistance R_{S} (Ω·cm) was 9.4 Ω·cm. The standard deviation σ was 0.26 Ω·cm. Thus, the coefficient of variation CV was relatively small at 2.7%.

Example 2: A cathode active material having a relatively small amount of gaps 321c, such as that shown in FIG. 3B, was used. This cathode active material will be referred to as “cathode active material B”. The particle composition was that of the cathode active material 321, and the particle structure was hollow secondary particles 321a. Accordingly, the oil absorption (ml/100 g) was 41 ml/100 g, which is approximately 2.5 times greater than that of the simulated primary particles 132a in Example 1. This means that the cathode active material B of Example 2 had the cavities 321d and the gaps 321c.

In Example 2, the average value of the coating resistance R_{S} (Ω·cm) was 9.1 Ω·cm. The standard deviation σ was 0.62 Ω·cm. Thus, the coefficient of variation CV was 6.8%, which is greater than Example 1.

Example 3: A cathode active material having a relatively large amount of gaps 321c, such as that shown in FIG. 3C, was used. This cathode active material will be referred to as “cathode active material C”. The particle composition was that of the cathode active material 321, and the particle structure was hollow secondary particles 321a. Accordingly, the oil absorption (ml/100 g) was 47 ml/100 g, which is even greater than that of Example 2. This means that the cathode active material C of Example 3 had a greater amount of cavities 321d and the gaps 321c than the cathode active material B of Example 2.

In Example 3, the average value of the coating resistance R_{S} (Ω·cm) was 20.5 Ω·cm. The standard deviation σ was 1.36 Ω·cm. Thus, the coefficient of variation CV was 6.6%, which is greater than Example 1.

Example 4: A mixture of the cathode active material B, having a relatively small amount of gaps 321c such as that shown in FIG. 3B, and the cathode active material C, having a relatively large amount of gaps 321c such as that shown in FIG. 3C, was used. This cathode active material will be referred to as “cathode active material B+C”. The particle composition was that of the cathode active material 321, and the particle structure was hollow secondary particles 321a. Accordingly, the oil absorption (ml/100 g) was 46 ml/100 g, which is an intermediate value between Examples 2 and 3. This indicates that the cathode active material B+C had properties between the cathode active material B of Example 2 and the cathode active material C of Example 3.

In Example 4, the average value of the coating resistance R_{S} (Ω·cm) was 14.8 Ω·cm. This indicates that the cathode active material B+C had properties between the cathode active material B of Example 2 and the cathode active material C of Example 3.

The standard deviation σ was 5.78 Ω·cm. Thus, the coefficient of variation CV was 39.1%, which is extremely large compared to Examples 2 and 3.

### Evaluation of Coefficient of Variation CV

FIG. 10 is a diagram illustrating the coefficients of variation CV indicating variability of the cathode active materials 321 and the simulated primary particles 132a. The simulated primary particles 132a formed of alumina in Example 1 had relatively small variability from its production stage. The coefficient of variation CV was 2.7%.

In contrast, the cathode active material B of Example 2 had a greater coefficient of variation CV of 6.8%, and the cathode active material C of Example 3 had a greater coefficient of variation CV of 6.6%. The variability was increased when the primary particles 321b were fired to prepare the secondary particles 321a of the cathode active material B. The cathode active material B+C of Example 4 was the mixture of the cathode active material B and the cathode active material C. The cathode active material B, having the coating resistance R_{S} of 9.1 Ω·cm, and the cathode active material C, having the coating resistance R_{S} (Ω·cm) of 20.5 Ω·cm, had different effects at different locations, and thereby increased the standard deviation σ to 5.78. The oil absorption (ml/100 g) and the coating resistance R_{S} (Ω·cm) were approximately the averages of those of Examples 2 and 3. As shown in FIG. 10, the coefficient of variation CV became relatively large at 39.1% due to the variability between the cathode active material B, having a relatively small coating resistance R_{S} (Ω·cm), and the cathode active material C, having a relatively large coating resistance R_{S} (Ω·cm)

### Optimization of Mixture Mass Ratio R (wt %) of Conductive Material 132b to Simulated Primary Particle 132A

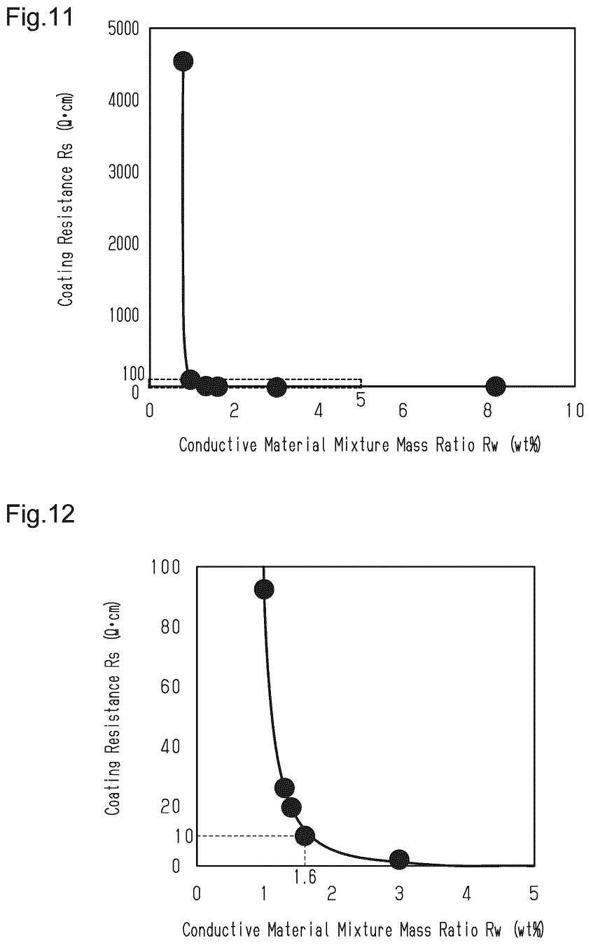

FIG. 11 is a graph showing a relationship of the coating resistance R_{S} (Ω·cm) of the test coating 132 and changes in the mixture mass ratio R_{W} (wt %) of the conductive material 132b to the simulated primary particles 132a. As shown in FIG. 11, when changing the mixture mass ratio R_{W} (wt %) of the conductive material 132b to the simulated primary particles 132a from zero, the initial coating resistance R_{S} (Ω·cm) was extremely high. Then, the coating resistance R_{S} (Ω·cm) suddenly decreased in a range in which the mixture mass ratio R_{W} (wt %) is approximately 1 to 2. This is most likely because the amount of the conductive material 132b reached a sufficient amount to form a conductive network between adjacent simulated primary particles 132a.

FIG. 12 is a graph enlarging part (0≤R_{W}≤5, 0≤R_{S}≤100) of the graph shown in FIG. 11. As shown in FIG. 12, the slope of the graph changes greatly within the range in which “0≤R_{W}≤5” and “0≤R_{S}≤100” are satisfied. Accordingly, the mixture mass ratio R_{W} (wt %) of the conductive material 132b to the simulated primary particles 132a was varied in a range of 0.8 to 3 wt % to check changes in the coating resistance R_{S} (Ω·cm) of the test coating.

It was found that the conductive material 132b may be added such that the mixture mass ratio R_{W} (wt %) of the conductive material 132b to the simulated primary particles 132a is between approximately 1 wt % and 3 wt %, inclusive. In particular, the graph of FIG. 12 indicates that the dispersibility of the conductive material 132b changed in a range near 1.6 wt %, at which the curvature of the graph was maximum. More specifically, when the mixture mass ratio R_{W} (wt %) was greater than 3 wt %, the coating resistance R_{S} (Ω·cm) did not change. This indicates that the mixture mass ratio R_{W} (wt %) of the conductive material 132b was excessive. In contrast, when the mixture mass ratio R_{W} (wt %) was less than 1 wt %, the mixture mass ratio R_{W} (wt %) of the conductive material 132b was insufficient to form a conductive network. These results show that the conductive material 132b may be added such that the mixture mass ratio R_{W} (wt %) of the conductive material 132b to the simulated primary particles 132a is between approximately 1 wt % and 3 wt %, inclusive.

### Operation of the Present Embodiment

When measuring the conductivity of the conductive material 322 contained in the cathode plate 3 of the completed lithium-ion rechargeable battery 1, such as that shown in FIG. 5B, the conductivity of the conductive material 322 is affected by the conductivity of the cathode active material 321. Further, the cavities 321d and the gaps 32c formed in the cathode active material 321, such as those shown in FIG. 3, change the density of the conductive material 322 in the binder 323. Furthermore, the conductivity of the conductive material 322 is affected by the conductive cathode substrate 31. Therefore, regardless of how accurately the coating resistance R_{S} (Ω·cm), corresponding to the surface resistance of the cathode plate 3, is measured, the conductivity and dispersibility of the conductive material 322, the formation of conductive network, and the like may not be evaluated correctly.

Accordingly, in the simulated cathode plate 103 shown in FIG. 5A, the cathode active material 321, which affects the measurement, is replaced with the insulative simulated primary particles 132a without changing other mechanical structures. Further, the cathode substrate 31 is replaced with the insulative simulated substrate 131 without changing other mechanical structures.

This allows for correct evaluations of the conductivity, dispersibility, and network formation of the conductive material 322 in a completed lithium-ion rechargeable battery 1.

Further, an appropriate amount of conductive material 322 is obtained through experiments using the simulated cathode plate 103.

### Advantages of the Present Embodiment

(1) The method for evaluating the conductive material 322 for use in the lithium-ion rechargeable battery 1 of the present embodiment accurately evaluates the conductivity and dispersibility of the conductive material 322.
(2) In the present embodiment, the lithium-ion rechargeable battery 1 includes the cathode plate 3 in which the cathode mixture layer 32, containing the cathode active material 321 and the conductive material 322, is formed on the cathode substrate 31. Accordingly, the method for evaluating the conductive material 322 for use in the lithium-ion rechargeable battery 1 allows the amount of the conductive material 322 to be set appropriately when manufacturing the lithium-ion rechargeable battery 1, which is the final product.
(3) The simulated cathode plate 103 includes the simulated primary particles, which are formed of an insulative material that simulates the cathode active material 321 of the lithium-ion rechargeable battery 1. This accurately reproduces the dispersion state of the conductive material 322 or the like, thereby allowing for accurate evaluation of the conductivity and dispersibility of the conductive material 322.
(4) Further, in the simulated cathode plate 103, the test coating paste is applied to the simulated substrate 131 that simulates the cathode substrate 31 of the lithium-ion rechargeable battery 1, and the applied paste is dried to prepare the test coating 132 containing the simulated primary particles 132a. This allows for accurate evaluation of the conductivity and dispersibility of the conductive material 322 without being affected by the conductivity of the cathode substrate 31.
(5) The coating resistance R_{S} (Ω·cm), which corresponds to the surface resistance of the test coating 132, is measured. This allows for accurate evaluation of the conductivity and dispersibility of the conductive material 322.
(6) The mixture volume ratio Rᵥ (vol %) of the conductive material 132b to the simulated primary particles 132a in the test coating paste is set in accordance with the mixture volume ratio R_{V} (vol %) of the cathode active material 321 to the conductive material 322. This accurately reproduces the operation of the conductive material 132b of the lithium-ion rechargeable battery 1 in the simulated cathode plate 103.
(7) The mixture mass ratio R_{W} (wt %) of the conductive material 132b to the simulated primary particles 132a is set in accordance with a range of a graph, which shows the coating resistance R_{S} (Ω·cm) of the test coating and changes in the mixture mass ratio R_{W} (wt %), the range including a part at which the curvature of the graph is maximum. This allows for accurate analysis of the operation of the conductive material 132b.

Through the experiments conducted by the inventors of the present disclosure, it was found that the appropriate mixture mass ratio R_{W} (wt %) of the conductive material 132b to the simulated primary particles 132a is between 1 wt % and 3 wt %, inclusive. This obtains the appropriate amount of the conductive material 132b.
(8) The average particle diameter D_{S} (d50) (μm) of the simulated primary particles 132a is set to be substantially the same as that of the particles of the cathode active material 321 in the lithium-ion rechargeable battery 1. This accurately reproduces the operation of the conductive material 132b of the lithium-ion rechargeable battery 1 in the simulated cathode plate 103.

Through the experiments conducted by the inventors of the present disclosure, it was analyzed that the appropriate average particle diameter D_{S} (d50) (μm) of the simulated primary particles is between 0.1 μm and 50 μm, inclusive.
(9) The test coating 132 is set to have a thickness (μm) that is the same as a thickness (μm) of the cathode mixture layer 32 of the lithium-ion rechargeable battery 1. This accurately reproduces the operation of the conductive material 132b of the lithium-ion rechargeable battery 1 in the simulated cathode plate 103.
(10) In the present embodiment, the cathode active material 321 includes the secondary particles of a lithium transition metal oxide. In such a case, the present embodiment effectively solves the technical problems. In addition, the present embodiment may be applied in a preferred manner when the conductive material 322 is formed of a fibrous carbon.
(11) The simulated primary particles 132a are formed of alumina. Alumina has a high insulating property and is mechanically stable. Thus, unnecessary electrochemical reactions will not occur. Preferably, the simulated substrate 131 is formed of an insulative material. In the present embodiment, the simulated substrate 131 is formed by a PET film. The PET film also has a high insulating property and is mechanically stable. Thus, unnecessary electrochemical reactions will not occur. Such configurations allow for accurate evaluation of the conductivity and dispersibility of the conductive material 322.
(12) In the conductive material 322, 132b, the average diameter D_{C} (d50) (nm) is between 1 nm and 100 nm, inclusive, and the average length L_{C} (d50) (nm) is between 100 nm and 10000 nm, inclusive. The material having such configurations is likely to be affected by the shape of the particles of the cathode active material 321. Accordingly, the present embodiment allows for accurate evaluation of the conductivity and dispersibility of such a conductive material 322.

### Modified Examples

The present embodiment is merely an example and is not intended to limit the present disclosure. The present disclosure may be modified as follows, and the modifications may be optimized by one skilled in the art.

In the present embodiment, the cathode active material 321 has a hollow structure including the gaps 321c and the cavity 321d. Alternatively, the present disclosure may be applied to the cathode active material 321 that is solid and does not have the gaps 321c or the cavity 321d. In such a case, CNT will not enter the gaps 321c or the cavity 321d. If the particles of the cathode active material 321 have rough surfaces or are uneven in size, the simulated primary particles 132a of the present disclosure may be used to evaluate the conductivity and dispersibility of the conductive material at a higher accuracy.

In the same manner, the electrode plate is not limited to the cathode plate 3 and may be the anode plate 2. For example, conductive paths may be examined when the anode active material of the lithium-ion rechargeable battery 1 includes graphite, silicon, or the like.

Furthermore, the rechargeable battery is not limited to a lithium-ion rechargeable battery and may be other types of non-aqueous electrolyte solution rechargeable battery, as well as other types of rechargeable battery, such as an alkaline rechargeable battery.

In the present embodiment, the conductive material 322, 132b includes a fibrous carbon, specifically, carbon nanotubes (CNT). Alternatively, other types of conductive material may be used, for example, carbon microfibers or granular acetylene black (AB).

The drawings schematically illustrate the method for evaluating the conductive material for use in the lithium-ion rechargeable battery of the present embodiment, and do not necessarily depict actual quantities, shapes, or dimensions.

Quantities, dimensions, and other numerical values and ranges, shapes, materials, or the like are merely examples and are not intended to limit the present disclosure. Such parameters and configurations may be appropriately optimized by one skilled in the art.

The procedure of the method for evaluating the conductive material for use in the lithium-ion rechargeable battery is an example. The order of steps may be modified, and steps may be added or omitted.

It should be apparent to one skilled in the art that the present disclosure may be embodied in many other specific forms without departing from the spirit or scope of the claims.

Various changes in form and details may be made to the examples above without departing from the spirit and scope of the claims and their equivalents. The examples are for the sake of description only, and not for purposes of limitation. Descriptions of features in each example are to be considered as being applicable to similar features or aspects in other examples. Suitable results may be achieved if sequences are performed in a different order, and/or if components in a described system, architecture, device, or circuit are combined differently, and/or replaced or supplemented by other components or their equivalents. The scope of the disclosure is not defined by the detailed description, but by the claims and their equivalents. All variations within the scope of the claims and their equivalents are included in the disclosure.

What is claimed is:

1. A method for evaluating a conductive material for use in a rechargeable battery, the rechargeable battery including an electrode plate in which a mixture layer, containing an active material and the conductive material, is formed on a substrate, the method comprising:
preparing a paste containing simulated primary particles and the conductive material, the simulated primary particles being formed of an insulative material that simulates the active material of the rechargeable battery;
applying the prepared paste to a simulated substrate that simulates the substrate of the rechargeable battery, and drying the applied paste to prepare a test coating containing the simulated primary particles; and
measuring a coating resistance R_{S} (Ω·cm), corresponding to a surface resistance of the test coating, to evaluate the conductive material.

2. The evaluation method according to claim 1, wherein a mixture volume ratio Rᵥ (vol %) of the conductive material to the simulated primary particles in the paste is set in accordance with a mixture volume ratio Rᵥ (vol %) of the conductive material to the active material.

3. The evaluation method according to claim 1, wherein a mixture mass ratio R_{W} (wt %) of the conductive material to the simulated primary particles is set in accordance with a range of a graph, the graph showing the coating resistance R_{S} (Ω·cm) of the test coating and changes in the mixture mass ratio R_{W} (wt %), the range including a part at which a curvature of the graph is maximum.

4. The evaluation method according to claim 1, wherein, when a mixture mass ratio of the conductive material to the simulated primary particles is represented by a mixture mass ratio R_{W} (wt %), the mixture mass ratio R_{W} (wt %) is between 1 wt % and 3 wt %, inclusive.

5. The evaluation method according to claim 1, wherein an average particle diameter D_{S} (d50) (μm) of the simulated primary particles is substantially the same as that of particles of the active material of the rechargeable battery.

6. The evaluation method according to claim 1, wherein an average particle diameter D_{S} (d50) (μm) of the simulated primary particles is between 0.1 μm and 50 μm, inclusive.

7. The evaluation method according to claim 1, wherein the test coating has a thickness (μm) that is the same as a thickness (μm) of the mixture layer of the rechargeable battery.

8. The evaluation method according to claim 1, wherein the active material includes secondary particles formed by aggregated primary particles.

9. The evaluation method according to claim 1, wherein the conductive material is formed of a fibrous carbon.

10. The evaluation method according to claim 1, wherein the simulated primary particles are formed of alumina.

11. The evaluation method according to claim 1, wherein the simulated substrate is formed of an insulative material.

12. The evaluation method according to claim 11, wherein the simulated substrate is formed by a PET film.

13. The evaluation method according to claim 1, wherein an average diameter D_{C} (d50) (nm) of the conductive material is between 1 nm and 100 nm, inclusive.

14. The evaluation method according to claim 1, wherein an average length L_{C} (d50) (nm) of the conductive material is between 100 nm and 10000 nm, inclusive.

15. The evaluation method according to claim 1, wherein the rechargeable battery is a lithium-ion rechargeable battery.

16. The evaluation method according to claim 15, wherein the electrode plate is a cathode plate.
